(19) United States
(12) Patent Application Publication
(10) Pub. No.: US 2010/0063525 A1
(43) Pub. Date: Mar. 11, 2010

(54) Ultrasonic shears force limiting

(76) Inventors: Jean Michael Beaupre, Alexandria, KY (US); Jason Andrew Stivers, Cincinnati, OH (US)

Correspondence Address: Jean M. Beaupre, Suite 412, 4480 Lake Forest Dr., Cincinnati, OH, 45242, US

(21) Appl. No.: 12/584,397

(22) Filed: Sep. 4, 2009

Related U.S. Application Data
(60) Provisional application No. 61/191,181, filed on Sep. 5, 2008.

(51) Int. Cl. A61B 17/32

(52) U.S. Cl. 606/169

(57) ABSTRACT

The present invention relates to an improved arrangement for limiting the force applied to tissue by a mounted pivotal clamp arm of the present clamp coagulator apparatus and providing the appropriate amount of force to correctly positioned tissue. The arrangement is desirably economical in configuration, and cooperates with the associated pivotal clamp arm to position and maintain the clamp arm in substantial alignment with an associated end-effector, notwithstanding normal manufacturing tolerances of the components.

### RELATED APPLICATIONS

This application hereby claims the priority of U.S. Provisional Application 61/191,181 filed on Sep. 5, 2008. U.S. Provisional Application 61/191,181 and US patent application for Improved Jaw filed on Sep. 4, 2009, US patent application for Improved Tissue Pad filed on Sep. 4, 2009, and US patent application for Ultrasonic Shears Actuating Mechanism filed on Sep. 4, 2009 are incorporated by reference.

### TECHNICAL FIELD

The present invention relates generally to ultrasonic surgical devices, and more particularly to ultrasonic surgical clamp coagulator apparatus for coagulating and/or cutting tissue.

### BACKGROUND OF THE INVENTION

Ultrasonic surgical instruments are finding increasingly widespread application in surgical procedures by virtue of the unique performance characteristics of such instruments. Depending upon specific instrument configurations and operational parameters, ultrasonic surgical instruments can provide substantially simultaneous cutting of tissue and hemostasis by coagulation, minimizing patient trauma. In some ultrasonic instruments the cutting action is typically effected by an end-effector at the distal end of the instrument, with the end-effector transmitting ultrasonic energy to tissue brought into contact therewith. Ultrasonic instruments of this nature can be configured for open surgical use, or laparoscopic or endoscopic surgical procedures.

Ultrasonic surgical instruments have been developed that include a clamp mechanism to press tissue against the end-effector of the instrument in order to couple ultrasonic energy to the tissue of a patient. Such an arrangement (sometimes referred to as an ultrasonic shears, ultrasonic clamp coagulator, or an ultrasonic transector) is disclosed in U.S. Pat. No. 5,322,055, incorporated herein by reference.

### SUMMARY OF THE INVENTION

The present invention provides an ultrasonically-actuated surgical instrument for cutting/coagulating tissue, including loose and unsupported tissue, wherein the ultrasonic actuated blade is employed in conjunction with a clamp for applying a compressive or biasing force to the tissue against the blade. The present invention provides the foregoing features, in one embodiment hereof, as an ultrasonic clamp coagulator accessory for a standard ultrasonic surgical system wherein the instrument may be particularly adapted for endoscopic surgery.

A standard ultrasonic surgical system comprises essentially a generator, which contains a power source for generating an ultrasonic frequency electrical drive sinusoidal waveform such as described in U.S. Pat. Nos. 5,026,387 and 6,063,050 (incorporated herein by reference) and a handpiece, containing a transducer for converting such electrical signal into longitudinal mechanical vibration for coupling to a blade assembly. Examples of suitable transducers include piezoceramic transducers as described in U.S. Pat. No. 7,285,895 (incorporated herein by reference), magnetostrictive transducers, or other means of producing ultrasonic vibration.

Examples of generators include Ethicon Endo-Surgery Generator 300 or Generator G-110 and the Covidien AutoSonix Generator Box. Examples of transducers, sometimes called handpieces, include Ethicon Endo-Surgery HP054 or HPBLUE and Covidien AutoSonix™ Transducer.

The clamp coagulator accessory adapts this standard ultrasonic unit for use in conjunction with a clamp assembly whereby tissue, particularly loose tissue, may be clamped between a clamping jaw and the blade for cutting and coagulating the tissue.

In one embodiment, an ultrasonic surgical apparatus is configured to permit selective cutting, coagulation, and/or clamping of tissue during surgical procedures. The apparatus includes a pivoting clam arm which may be selectively pivoted towards and ultrasonic end effector. During use, tissue may be compressed against the ultrasonic end-effector by the clam arm, thereby allowing the tissue to be clamped, cut, and/or coagulated.

The apparatus may be configured such that the pivotal clamp arm of the clamping mechanism is maintained in substantial alignment with the ultrasonic end-effector. Recognizing that normal manufacturing tolerances can result in misalignment of the clamp arm and end-effector, one embodiment of the present invention includes a clamp arm mounting arrangement which provides a “self-centering” action which maintains the clamp arm in the desired alignment with the ultrasonic end-effector. This desired alignment is achieved even when components of the apparatus, including the pivotal clamp arm, are dimensioned within normal manufacturing tolerances.

In accordance with one embodiment, the present surgical apparatus includes a housing, and an inner tubular sheath having a proximal end joined to the housing. The inner tubular sheath may be joined with the housing in a manner which allows for rotation of the inner tubular sheath relative to the housing. An outer actuating member is reciprocably positioned around the inner tubular sheath such that the outer actuating member may reciprocally move longitudinally along the inner tubular sheath. An operating lever may be mounted on the housing and configured to effect selective reciprocable movement of the outer actuating member with respect to the inner tubular member.

An ultrasonic waveguide, or blade, is positioned within the inner tubular sheath, and includes an end-effector extending distally of a distal end of the outer tubular sheath. In order to couple tissue with the ultrasonic end-effector, the apparatus includes a clamp arm pivotally mounted on the distal end of the inner tubular sheath for pivotal movement with respect to the end-effector. In this fashion, tissue can be clamped between the clamp arm and the end-effector for creating the desired ultrasonic effect on the tissue. The clamp arm is also operatively connected to the outer actuating member so that reciprocable movement of the outer actuating member pivotally moves the clamp arm with respect to the end-effector.

In one embodiment, a rotating member such as a spline knob may be mounted on the housing in order to allow the user to align the blade and other components. For example, notches may be located on the inside of a spline knob engage openings on the inner and outer tube and on the blade shaft to ensure rotational alignment of the said inner tube and outer tube with the blade. Said spline knob serves as a means of rotating said blade to achieve desired alignment. Said notches may be oriented with respect to the blade end-effector to adjust the orientation of the blade with respect to the clamp arm.

In one embodiment, the clamp is actuated by a scissor-like grip created by a thumb lever movably located on the under side of the handle housing and a finger grip located at the proximal end of the ultrasonic wave guide. Said thumb lever may be connected to a metal lever extending upwards towards the waveguide. The metal lever may be connected to a yoke assembly that engages the slideable outer tube, thereby allowing proximal and distal sliding movement of the thumb lever to slide the outer tube proximally and distally respectfully.

A pin may be received through a distal end portion of the outer tube to engage a flat or a curved camming portion of the proximal end of the clamp arm. Distal motion of the slideable outer tube creates a camming motion acting upon said clamp arm. Furthermore, the clamp arm may be pivotally mounted via two mounting pins located at opposite side of the proximal end of said clamp along the circumference near the center of the distal end of the non-slideable inner tube so that the motion of the pin on the camming surface results in an opening and closing of the jaw with respect to the ultrasonic blade.

This camming surface of the clamp arm may be distal or proximal to said clamp arm pivot, improving alignment between the clamp arm and blade. Thus, in one embodiment, by significantly reducing or eliminating relative motion between the inner tube and the blade, damage and failures of the blade seal can be reduced or eliminated.

In one embodiment, a yoke assembly may be provided and includes a force-opposing member that engages a pre-loaded force-limiting spring. When said movable thumb lever moves distally, moving the clamp arm into a clamped position, said metal lever engages the force-opposing member, engaging the force-limiting spring, thus preventing adverse forces from being applied to the jaw.

In accordance with one embodiment of the present invention, the outer tubular sheath includes a clamp arm mount, generally at the distal end thereof, on which the clamp arm is pivotally mounted. In order to maintain the clamp arm in the desired alignment with the associated end-effector, the clamp arm mount may engage the clamp arm, so as to provide a “self-centering” action in cooperation therewith. This engagement, which is accommodated by longitudinally parallel surfaces of the clamp arm and clamp arm mount, may accommodate normal manufacturing tolerances of the components, particularly the clamp arm, while maintaining the clamp arm in substantial alignment with the ultrasonic end-effector.

In accordance with one illustrated embodiment, the clamp arm mount may have a generally U-shaped cross-section. The clamp arm mount includes a pair of laterally spaced leg portions which engage the clamp arm. The longitudinal parallel surfaces guide the clamp arm while opening and closing to maintain the clamp arm in substantial alignment. Each leg portion may define a respective pivot opening for receiving an associated pivot pin for pivotal mounting of the clamp arm. The clamp arm may include a pair of integral pivot pins respectively positioned on laterally spaced portions of the clamp arm. The integral pivot pins are configured for respective pivotal mounting in the pivot openings defined by the leg portions of the clamp arm mount.

In one embodiment, the clamp arm holds or includes a tissue pad located substantially along the tissue side of the clamp arm, which acts as a clamping surface against the blade (i.e. the side facing the end effector of the blade). Said tissue pad may have a planar, concave, or convex tissue engagement surface. Said tissue pad may be adhered to said clamp arm by means of a glue or intermediate layer containing one or more adhesive surfaces. Said tissue pad may also attach mechanically to said clamp arm by means, for example, of molding said tissue pad into a shape with one or more columnar standoffs projecting from the tissue pad extending through the clamp arm and terminating on the opposite, outer surface of the clamp arm and comprising one or more features that are substantially larger than the columnar portion of the standoff, engaging the outer surface of the clamp arm securing the tissue pad to the clamp arm. Furthermore, the clamp arm may comprise indented features to accept said substantially larger features of said columnar standoffs, further securing the tissue pad to the clamp arm. Said tissue pad may also attach mechanically to said clamp arm via a substantially V-shaped or T-shaped slot located on the tissue engaging side of said clamp arm. Said tissue pad may comprise a substantially V-shaped or T-shaped projection that would engage said V-shaped or T-shaped slot. Furthermore, said tissue pad may comprise one or more curved tissue stop pads located proximally from the parallel tissue engaging surface of the tissue pad. Said curved tissue stop pads may curve from a direction parallel to the blade engaging surface of said tissue pad to a direction greater than 30 degrees from parallel and preferably substantially perpendicular to the orientation of the blade and act as an additional tissue grasping and manipulating surface.

Other features and advantages of the present invention will become readily apparent from the following detailed description, the accompanying drawings, and the appended claims.

### BRIEF DESCRIPTION OF THE DRAWINGS

FIG. 1 is a perspective view of an ultrasonic surgical instrument system.

FIG. 2 is a side view of one embodiment of ultrasonic shears disclosed herein.

FIG. 3 is a perspective view of the ultrasonic shears of FIG. 2.

FIG. 4 is a side section view of the ultrasonic shears of FIG. 2.

FIG. 5 is a detailed side view of the housing of the ultrasonic shears of FIG. 2, with the left housing removed.

FIG. 6 is a side view of the ultrasonic shears of FIG. 2.

FIG. 7 is a side view of one embodiment of the end-effector of the ultrasonic shears of FIG. 2.

FIGS. 8A and 8B are side section views of the end-effector of FIG. 7.

FIGS. 9A and 9B are perspective section views of the end-effector of the ultrasonic shears disclosed herein.

FIGS. 10A and 10B are perspective section views of the end-effector of the ultrasonic shears disclosed herein.

FIGS. 11 through 13 show the motion of the ultrasonic shears instrument including the force-limiting mechanism disclosed herein.

FIG. 14A is a perspective view of the end-effector, actuating tube, spline knob assembly disclosed herein.

FIG. 14B is a perspective view of the spline knob assembly with one half of the spline knob removed.

FIG. 14C is a side view of the spline knob assembly with one half of the spline knob removed.

FIGS. 15A and 15B are section views of the spline knob assembly disclosed herein.

FIGS. 16 and 17 are exploded perspective views of the ultrasonic shears instrument disclosed herein.

FIGS. 18A, 18B, and 18 C are side, bottom and isometric views of a clamp arm with tissue gripping feature

FIGS. 19A and 19B are side and isometric views of an end effector with clamp arm containing a compliance member.

FIGS. 20A and 20B are a side view and a perspective view, respectfully, of an ultrasonic instrument end effector including tissue pad securing features.

FIGS. 21A, 21B, and 21C are a side view, a top view, and a section view, respectfully, of a clamp arm including tissue pad securing features.

FIGS. 22A, 22B, and 22C are a side view, a top view, and a section view, respectfully, of a clamp arm including tissue pad securing features

FIGS. 23A and 23B are a side view and a perspective view, respectfully, of a clamp arm including tissue grasping features.

FIGS. 24A and 24B are a side view and a perspective view, respectfully, of a clamp arm including tissue pad securing features.

FIGS. 25A and 25B are a side view and a perspective view, respectfully, of a clamp arm including tissue grasping features

FIG. 26 is a cross section view of a clamp arm including tissue grasping features

FIG. 27 is a list of all elements described herein.

### DETAILED DESCRIPTION OF THE INVENTION

The present invention will be described in combination with ultrasonic instruments as described herein. Such description is exemplary only, and is not intended to limit the scope and applications of the invention.

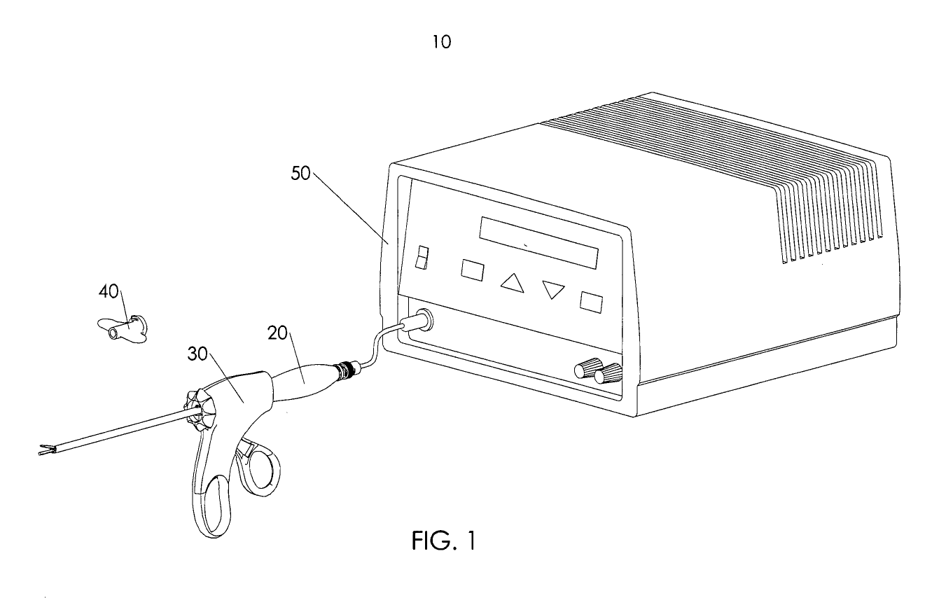

FIG. 1 illustrates one embodiment of an ultrasonic system 10 for coagulating and/or cutting tissue. Ultrasonic system 10 may comprise an ultrasonic signal generator 50, an ultrasonic transducer 20, ultrasonic surgical apparatus 30. In the embodiment shown in FIG. 1, ultrasonic surgical apparatus 30 is configured as ultrasonic shears for cutting and coagulating tissue. A torque tool 40 which may be used to secure ultrasonic shears 30 to ultrasonic transducer 20 is also shown in FIG. 1.

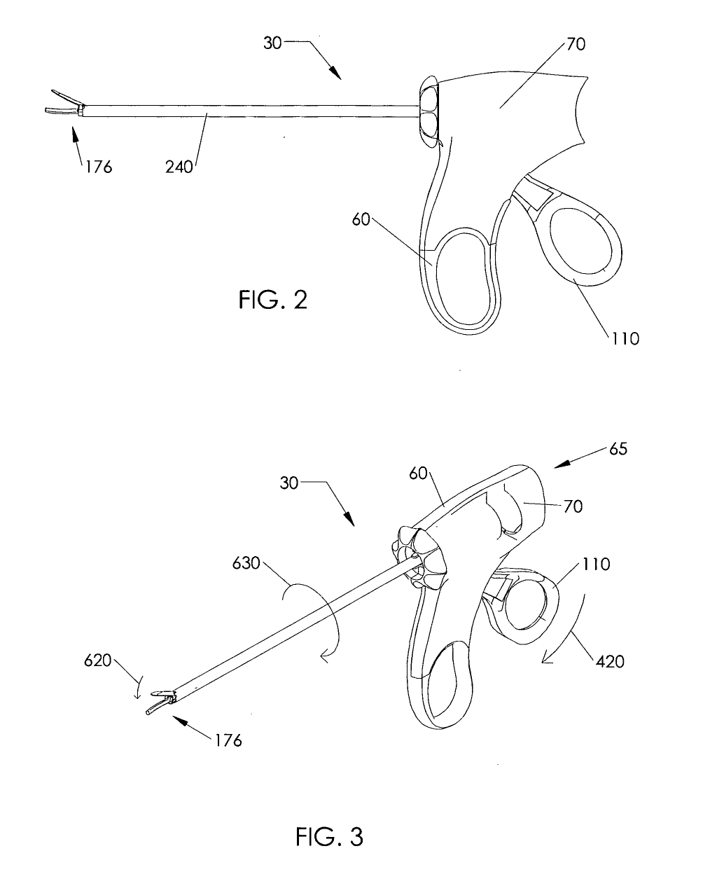

FIGS. 2 and 3 further illustrate one embodiment of ultrasonic shears 30. Ultrasonic shears 30 comprise a housing 65, which may include a right housing 60 and a left housing 70. Proximal of said housing is a movable thumb lever 110, the thumb lever distal motion 420 of which is shown. Rotational movement 630 is also shown allowing for alignment of the end effector 176 during use. Clamp arm closing motion 620 is illustrated and is resultant of said thumb lever distal motion.

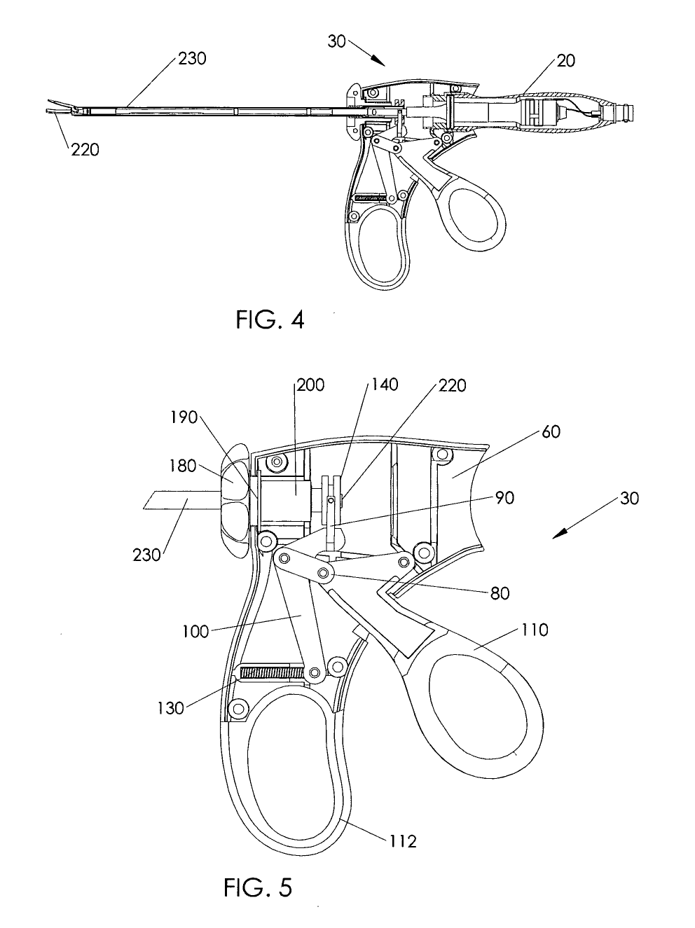

FIG. 4 is a partial section view of ultrasonic shears 30, illustrating the securing of ultrasonic transducer 20 onto the ultrasonic shears. In the embodiment shown, ultrasonic blade 220 is secured to transducer 20 using a threaded connection. This permits the transmission of ultrasonic vibration from ultrasonic transducer 20 to ultrasonic blade 220. Alternative connection means providing a secure interface between ultrasonic transducer 20 and ultrasonic blade 220 may also be used.

FIG. 5 illustrates the handle portion of shears 30 with left housing 70 hidden to reveal the inner workings. Shown is the right housing 60 of the ultrasonic shears 30, which includes finger grip 112. Finger grip 112 and thumb lever 110 create a scissor grip movably located on the under side of the right handle housing 60. Said thumb lever 110 connects to a linkage 80 operably connected to a yoke assembly 90 that engages the actuating outer tube 230, thereby allowing proximal lever motion 410 and distal lever motion 420 of the thumb lever 110 to slide the outer tube with a proximal motion 510 and distal motion 520 respectively (see FIG. 6). The yoke assembly 90 may include a force-opposing member 100 that engages a pre-loaded force-limiting spring 130. Drive flange 140 transfers force from said yolk assembly to the outer actuating tube 230. Spline knob 180 acts as a means of rotating shaft assembly 240 and thus ultrasonic blade 220 to achieve desired alignment. Sleeve 200 houses and compresses the distal portion of said spline knob 180. Washer 190 acts as a rotation and thrust bearing for shaft assembly 240 and prevents backlash.

FIG. 6 is a side view of the ultrasonic shears 30, illustrating the relationship between the motion 400 of thumb lever 110 relative to the outer actuating tube 230, clamp arm 150, tissue pad 170 and the ultrasonic blade 220. Proximal motion 410 of thumb lever 110 results in proximal motion 510 of outer actuating tube 230, which results in the opening motion 610 of the clamp arm 150 relative to the ultrasonic blade 220. Conversely, distal motion 420 of said thumb lever 110 results in distal motion 520 of said outer actuating tube 230, which results in the closing motion 620 of said clamp arm 150 and tissue pad 170 relative to said ultrasonic blade 220.

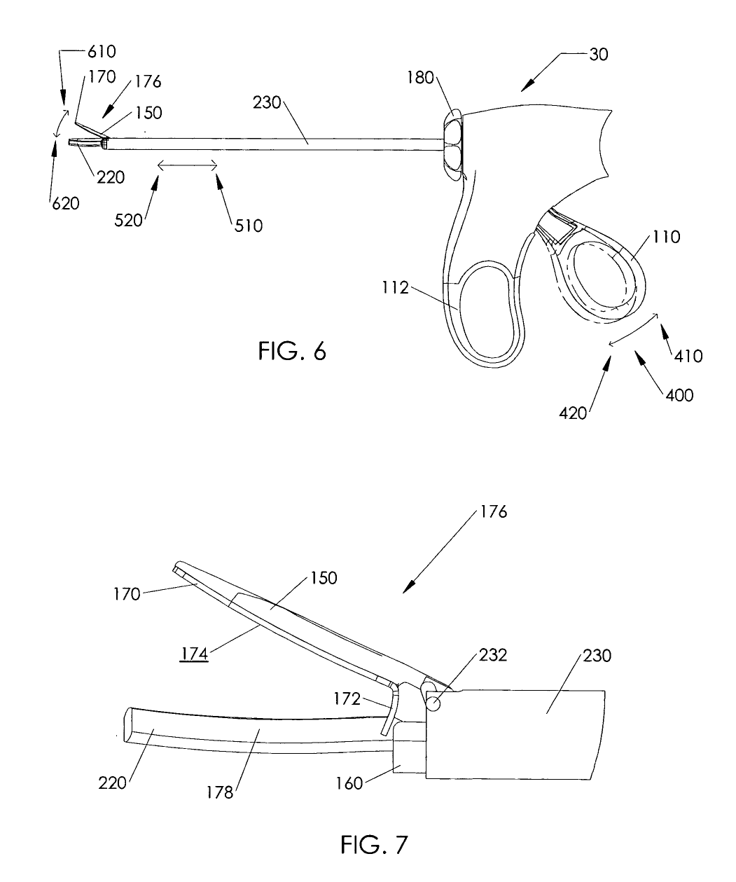

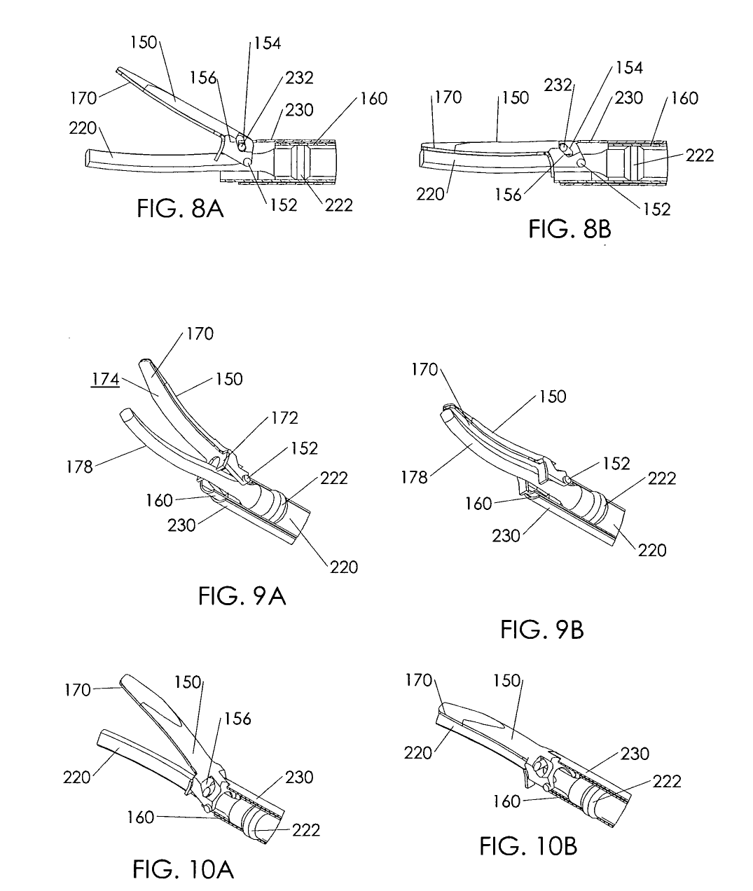

FIG. 7 is a side closeup view of the end-effector 176 of the ultrasonic shears 30. Outer actuating tube 230 operably connects to clamp arm 150 via actuating pin 232. Non-actuating inner tube 160 is shown extending distally from inside said outer actuating tube 230. Inner tube 160 remains stationary with respect to ultrasonic blade 220 and blade seal 222 (see FIG. 8A). Tissue pad 170 is shown connected to said clamp arm 150 to operably contact with ultrasonic blade 220 and tissue therebetween when in surgical use. Furthermore, said tissue pad 170 may comprise one or more tissue stop pads 172 located proximally from the blade engaging surface 174 of the tissue pad 170. The tissue stop pads 172 may curve from a direction parallel to the blade engaging surface 174 of said tissue pad 170 to a direction between 30 degrees and substantially perpendicular to the orientation of the ultrasonic blade 220 and act to position and manipulate tissue and may act as an initial barrier to prevent tissue from engaging undesired portions of the blade 220 or clamp arm 150 during surgical use. Blade engaging surface 174 may be convex and/or conformal to blade end effector 178.

In one embodiment, tissue stop pads 172 may engage tissue while clamp arm 150 is in the open position. As clamp arm 150 closes, tissue stop pads 172 force the tissue in contact with the tissue stop pads 172 distally and downward against ultrasonic blade 220. This stretches tissue across ultrasonic blade 200, creating tension in the tissue for use when cutting and/or coagulating. Tissue tension aids in the speed of cutting and coagulation.

FIGS. 8A and 8B are side partial section views of the end effector 176 of the ultrasonic shears. FIG. 8A shows the end effector 176 with the clamp arm 150 in the open position. FIG. 8B shows the end effector 176 with clamp arm 150 in the closed position. Clamp arm 150 rotatably attaches via pivot pin 152 to non-actuating inner tube 160. The axis of pivot pin 152 may be positioned above, below, or passing through the axis of ultrasonic blade 220. Clamp arm 150 pivots about pivot pin 152 when outer actuating tube 230 slides distally or proximally, engaging actuating pin 232 which is mounted at the substantially distal end of the outer actuating tube 230 and extending through cam slot 154 and operably engaging cam surface 156. Ultrasonic blade 220 extends through the interior of tube 160 and is engaged by tissue pad 170 which is connected to clamp arm 150 to facilitate clamping tissue between tissue pad 170 and ultrasonic blade 220. If tissue stop pads 172 are positioned near ultrasonic blade 220, they may perform a wiping action, clearing said ultrasonic blade of tissue upon opening and closing of clamp arm 150.

The profile and location of cam slot 154 and cam surface 156 may be selected to provide constant or variable mechanical advantage as actuating pin 232 moves distally or proximally. As clamp arm 150 rotates, the contact angle between cam surface 156 and actuating pin 232 provides a quantifiable mechanical advantage that can be chosen to meet the requirements for manipulating tissue for the position of clamp arm 150. The profile of cam surface 156 may be straight, contain one or more curves, or any combination thereof. Cam surface 156 may also include indentions or protuberances to give sensory feedback as actuating pin moves along the surface. Cam slot 154 may be placed distal or proximal to pivot pin 152.

In one embodiment, a steeper angle with respect to the motion of actuating pin 232 will provide faster clamp arm 150 closing speed with lower mechanical leverage, while a shallower angle will provide slower clamp arm 150 closing speed with higher mechanical leverage. When outer actuating tube 230 is positioned as shown in FIG. 8A, the contact angle is steep, providing faster closing speed than when outer actuating tube 230 is positioned as shown in FIG. 8B. However, the mechanical advantage is greater in FIG. 8B, allowing significant clamping force to be applied to tissue.

In one embodiment, actuating pin 232 may be mounted at the substantially distal end of an inner actuating tube and extending through cam slot 154 and operably engaging cam surface 156. Clamp arm 150 rotatably attaches via pivot pin 152 to non-actuating outer tube. Clamp arm 150 pivots about pivot pin 152 when inner actuating tube slides distally or proximally, engaging actuating pin 232.

FIGS. 9A, 9B, 10A, and 10B are alternate partial sectional views of said end effector 176 of said ultrasonic shears 30. Shown is blade seal 222, which does not move with respect to blade 220 and inner tube 160. Blade seal 222 may be bonded to ultrasonic blade 220 or inner tube 160. Alternately, blade seal 220 may be held in place through mechanical means. Reducing or eliminating the relative motion of blade seal 222 with respect to ultrasonic blade 220 and inner tube 160 allows for a tighter seal and reduces wear. This further reduces potential fluid migration along the shaft of blade 220 inside inner tube 160. Fluid along the shaft of blade 220 can produce unwanted and potentially dangerous heat as ultrasonic energy is damped out by the fluid. Reducing fluid migration reduces parasitic diversion of ultrasonic energy from blade 220 into waste heat, which can result in patient injury in some circumstances. By moving actuating tube 230 rather than inner tube 160, the risk of patient injury can be reduced. Seal integrity is further enhanced by locating blade seal 222 with respect to blade 220 and inner tube 160 during manufacture of ultrasonic shears 30.

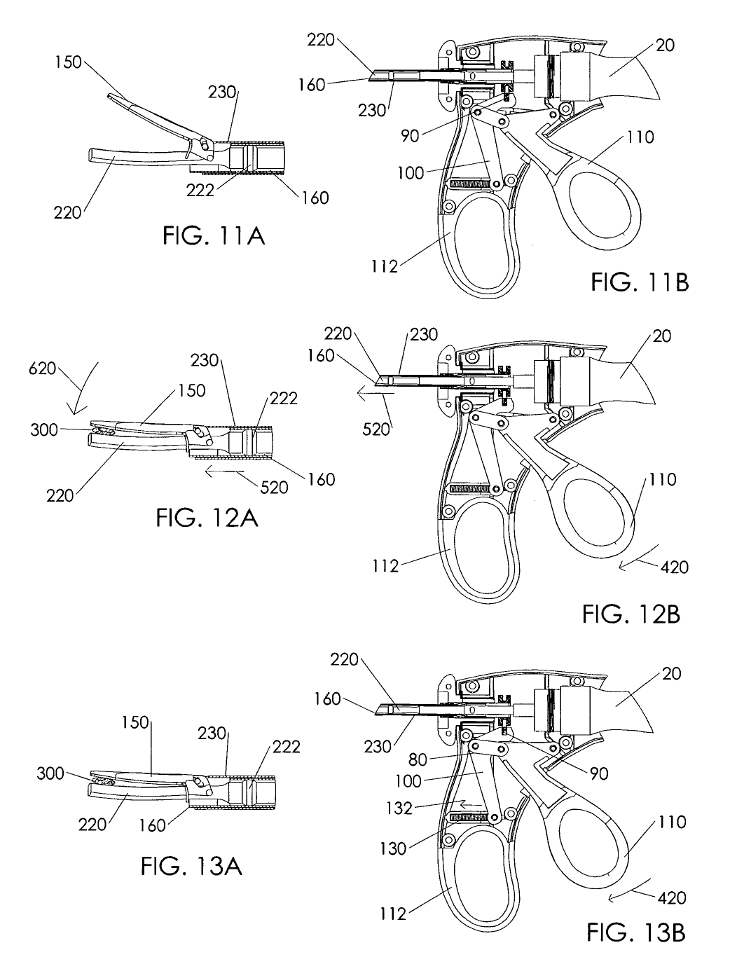

FIGS. 11A and 11B illustrate partial section views of the ultrasonic shears 30. The clamp arm 150 is actuated by a scissor-like grip created by a thumb lever 110 movably located on the under side of the right handle housing 60 and finger grip 112 located at the proximal end of the ultrasonic blade 220. Said thumb lever connects to a linkage 80 operably connected to yoke assembly 90 that engages the actuating outer tube 230, thereby allowing proximal and distal sliding movement of the thumb lever to slide the outer tube proximally and distally respectfully, resulting in the opening and closing movement of said clamp arm.

FIGS. 12A and 12B further illustrate the actuating motion of the ultrasonic instrument. Said elements described above actuate upon living tissue 300 in the manner described. Relative motion of finger grip 112 with respect to lever 110 produces motion in clamp arm 150 with respect to blade 220. In the embodiment shown, distal motion 420 of thumb lever 110 results in distal motion 520 in outer actuating tube 230 producing closing motion 620 of clamp arm 150 and tissue pad 170, thereby compressing tissue 300 against blade 220.

FIGS. 13A and 13B further illustrate the yoke assembly 90 which includes a force-opposing member 100 that engages a pre-loaded force-limiting spring 130. When slideable thumb lever 110 moves distally, moving said clamp arm 150 into a clamped position, linkage 80 engages the force-opposing member, engaging the force-limiting spring, thus preventing adverse forces from being applied to the clamp arm or the tissue 300 shown clamped between said clamp arm and said ultrasonic blade. Continued distal motion 420 on thumb lever 110 results in spring compression 132, limiting the force applied to tissue 300. By careful selection of the point of farthest travel by the said actuating tube and the preload of the said force-limiting spring 130, the tissue can be compressed and transected with a clamping force within a desirable range.

In one embodiment, force-limiting spring 130 is a helical spring. Force limiting spring 130 may also be any of the following types of springs: a cantilever, coil, conical, volute, leaf, V-spring, Belleville, disc, constant-force, gas, mainspring, elastomeric, washer, torsion, extension, wave or other deformable component.

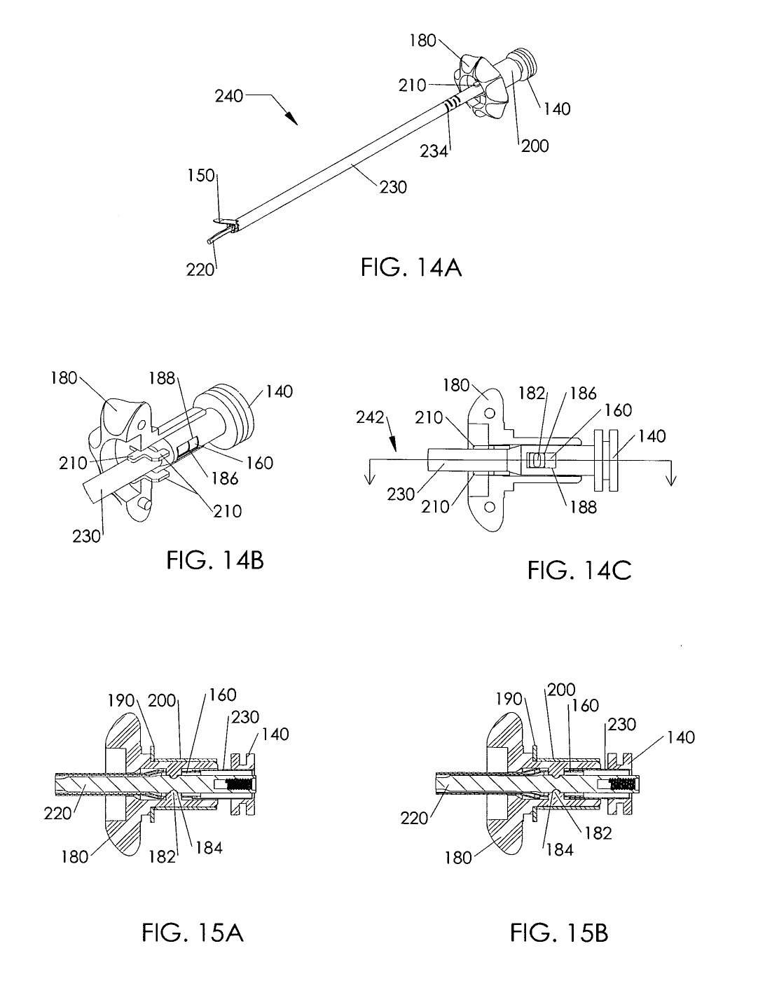

FIG. 14A illustrates the shaft assembly 240 of one embodiment of the ultrasonic shears apparatus. Drive flange 140 transfers force from the above-described yolk assembly to the outer actuating tube 230. Spline knob 180 acts as a means of rotating said shaft assembly, and thus ultrasonic blade 220 to achieve desired alignment. Sleeve 200 houses and compresses the distal portion of said spline knob. Torque tab 210 engages the torque tool 40 to secure or disconnect the ultrasonic shears instrument to the ultrasonic transducer 20. Compliance feature 234 is created by notches which allow the outer actuator tube 230 to compress when significant axial load is applied. Notches may alternate or form a spiral pattern. By careful selection of the point of farthest travel by the said actuating tube and the preload of the said compliance feature 234, the tissue can be compressed and transected with a clamping force within a desirable range.

FIGS. 14B and 14C are a perspective view and a side view, respectfully, of one embodiment of the spline knob assembly with washer 190, sleeve 200, blade 200, and one half of the spline knob 180 removed, showing torque tabs 210. Tabs 182 located on the inside of a spline knob engage inner tube openings 186 and outer tube openings 188 on the non-actuating inner tube 160 and outer actuating tube 230 and recesses 184 on the ultrasonic blade 220 to ensure rotational alignment of the said inner tube and outer tube with the blade. Said spline knob serves as a means of rotating said blade to achieve desired alignment. Said tabs and recesses are oriented with respect to the blade end-effector 178 to adjust the orientation of the blade end effector 178 to the clamp arm. Sleeve 200 acts to house said spline knob and the blade and tube assembly as well as secure washer 190, which acts to securely locate said shaft assembly within the ultrasonic shears instrument handle assembly. Sleeve 200 compresses said spline knob, compressing tabs 182 into recesses 184, substantially aligning the features. Cross section 242 intersects the assembly for purposes of illustration in FIGS. 15A and 15B.

FIGS. 15A and 15B illustrate cross sectional views of the spline knob assembly with outer actuating tube 230 in different positions.

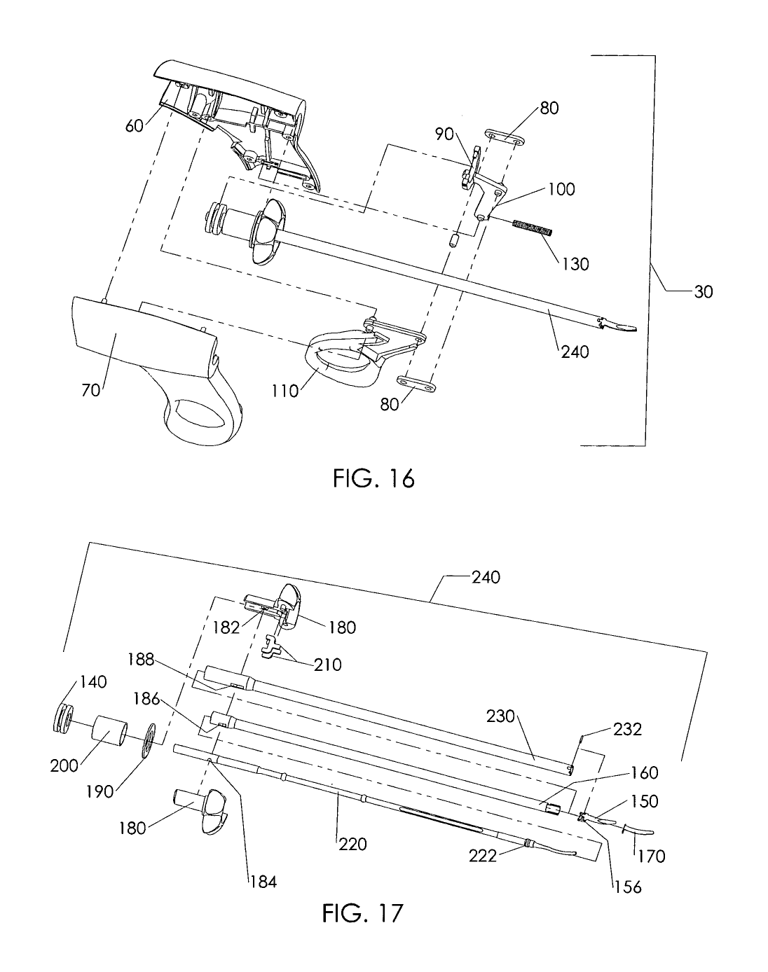

FIG. 16 is an exploded view of the ultrasonic shears apparatus showing some of the previously described components and subassemblies of one embodiment.

FIG. 17 is an exploded view of one embodiment of the ultrasonic shears shaft assembly showing components and features unobstructed by outer components of one embodiment.

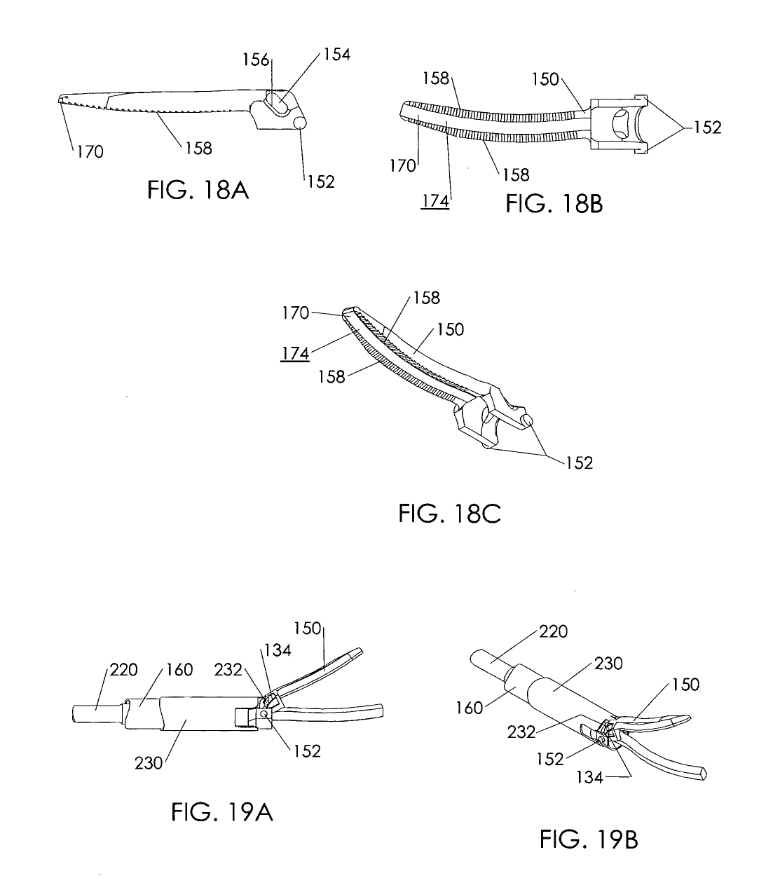

FIGS. 18A, 18B, and 18C illustrate one embodiment of a clamp arm 150 having integral tissue grip features 158. Clamp arm 150 and tissue pad 170 may be made from a metal and a polymer respectively. Tissue grip features 158 are designed to prevent tissue from slipping while being manipulated. Said tissue grip features may be any non-smooth surface, including but not limited to teeth, bumps, ridges, holes, and knurls. Tissue grip features 158 made from metal will withstand wear and damage better than equivalent features on a polymer tissue pad 170. Tissue pad 170 may be attached to said clamp arm and may or may not be designed to provide additional gripping force on tissue. The width of blade engaging surface 174 of tissue pad 170 may be the same or less than the width of clamp arm 150.

FIGS. 19A and 19B illustrate an embodiment of clamp arm 150 which includes an integrated compliance member 134. Said compliance member is operably connected to an actuator such as the outer actuating tube 230 and to said clamp arm. Said compliance member deforms when force is applied by said outer actuating tube, reducing the closing motion 620 when resistance is met. Said outer actuating tube is prevented from traveling beyond a set point, limiting the force that may be applied to said clamp arm. Said compliance member may be preloaded to prevent deformation until the said applied force is above a threshold. By careful selection of the point of farthest travel by the said actuating tube and the preload of the said compliance member 134, the tissue can be compressed and transected with a clamping force within a desirable range.

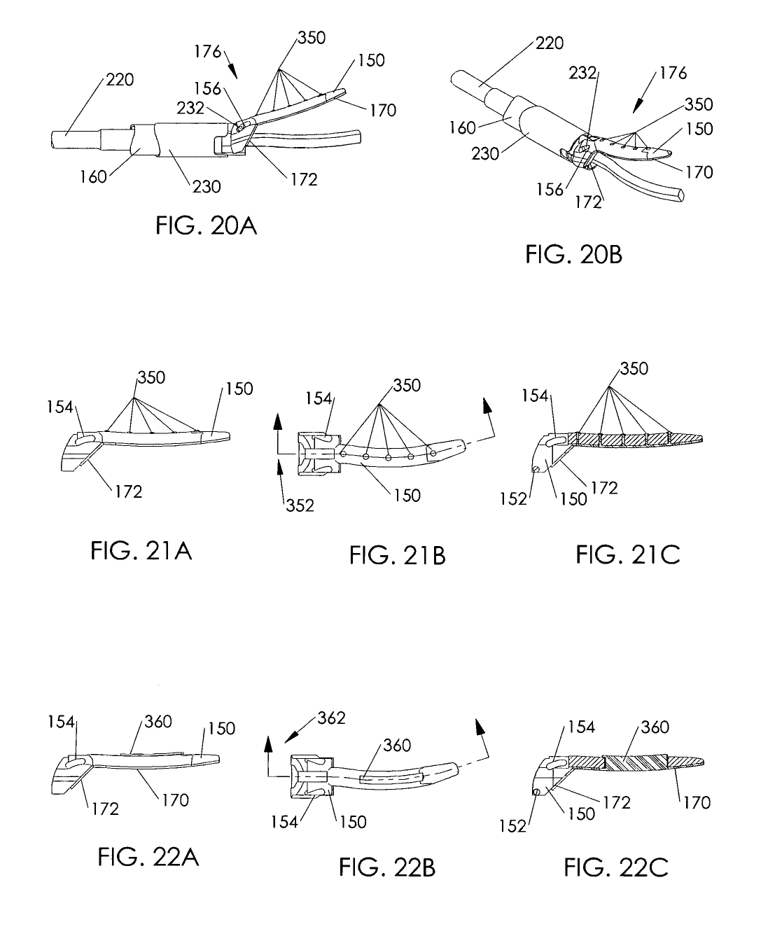

FIGS. 20A and 20B illustrate an embodiment of an end effector 176 with a clamp arm 150 with a tissue pad 170 connected to said clamp arm via one or more tissue pad rivets 350 extending through the cross-sectional area of said clamp arm, terminating on the side opposite of tissue interaction of said tissue pad of said clamp arm. Said tissue pad rivets, for example, can be molded or inserted through said clamp arm and then heat processed so that said tissue pad rivets form a substantially larger, opposing surface on said side opposite of tissue interaction.

FIGS. 21A through 21C illustrate an embodiment of clamp arm 150 which includes tissue pad rivets 350. Cross section 352 is shown in FIG. 21C.

FIGS. 22A through 22C illustrate an embodiment of clamp arm 150 which includes a tissue pad 170 including tissue pad connection member 360 extending substantially longitudinally along the length of said clamp arm and through the cross-sectional area of said clamp arm, terminating on the side opposite of tissue interaction of said tissue pad of said clamp arm. Said tissue pad connection member forms an enlarged, substantially flattened, opposing surface on said side opposite of tissue interaction. Cross section 362 is shown in FIG. 22C.

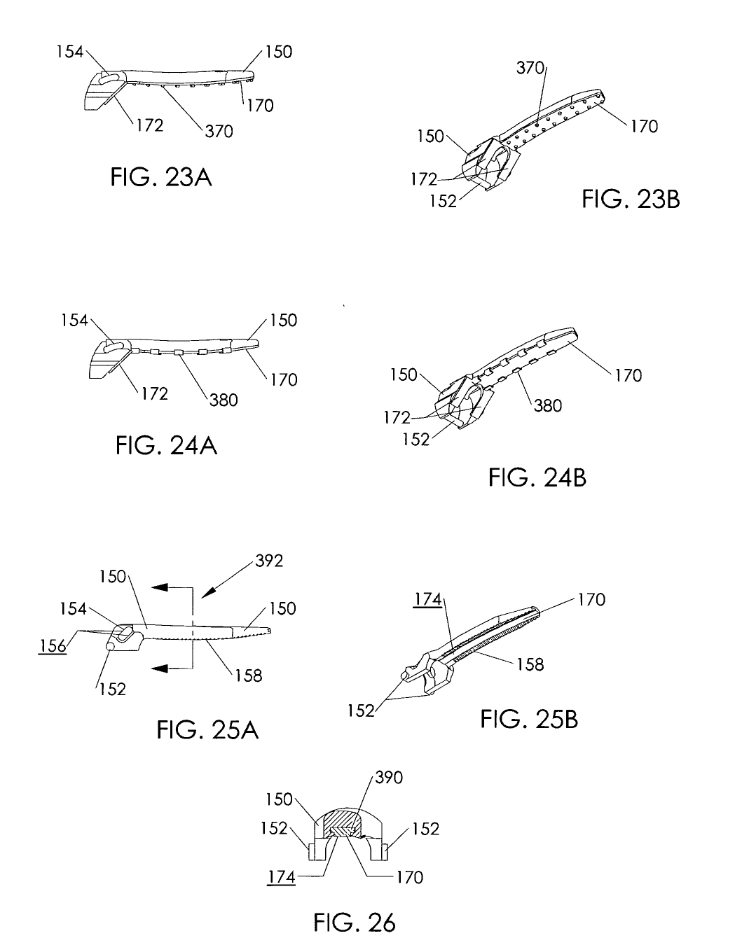

FIGS. 23A and 23B illustrate an embodiment of clamp arm 150 which includes a tissue pad 170 including clamp arm projections 370 located on the surface of clamp arm 150. Said clamp arm projections project through tissue pad 170 and may secure it through friction or mechanical interference. Said clamp arm projections also may interact with tissue creating an improved means of gripping tissue.

FIGS. 24A and 24B illustrate an embodiment of clamp arm 150 which includes a tissue pad 170 secured to said clamp arm via one or more opposing tissue pad securing tabs 380 located along the length of said clamp arm. Said tissue pad securing tabs also may interact with tissue creating an improved means of gripping tissue.

FIGS. 25A and 25B illustrate an embodiment of clamp arm 150 which includes tissue pad 170 located between tissue grip features 158 located along the length of said clamp arm.

FIG. 26 shows cross section 392. The width of blade engaging surface 174 of tissue pad 170 may be the same or less than the width of clamp arm 150. Tissue pad 170 is held in place by means of a slot feature 390.

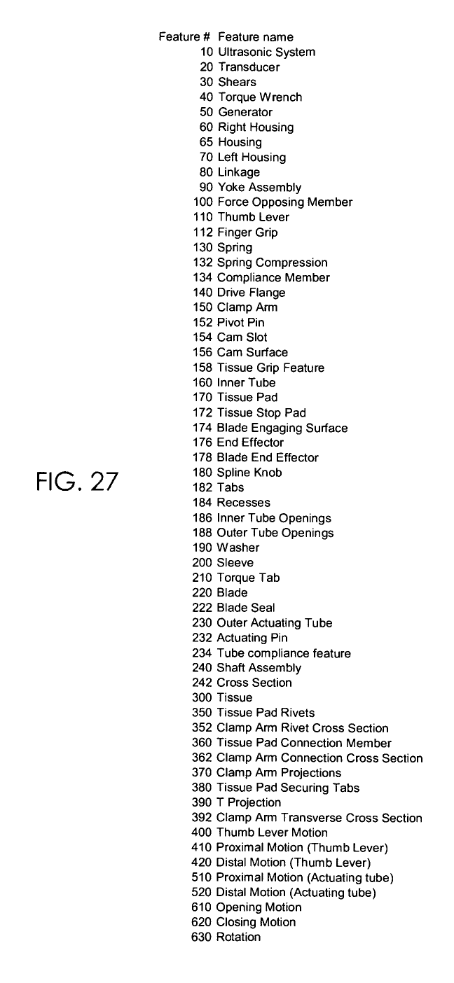

FIG. 27 is a list of all elements described herein.

Thus, the described embodiments are to be considered in all aspects only as illustrative and not restrictive, and the scope of the invention is, therefore, indicated by the appended claims rather than the foregoing description. All changes that come within the meaning and range of equivalency of the claims are to be embraced within their scope.

1. An ultrasonic surgical apparatus, comprising;
a housing;
an outer tube carried by said housing, said outer tube having distal and proximal ends;
an inner member carried by said housing, said inner member having distal and proximal ends;
a blade member carried by said housing, said blade member having an elongate shaft and an end effector located at a distal end of the blade member, said blade shaft positioned at least partially within the interior of said outer tube;
a clamp arm connected to said outer tube adjacent the distal end of the blade member, said clamp arm configured for selective pivotal movement towards the end effector of said blade member such that, during use, pivotal movement of the clamp arm toward the end effector of the blade member may be used to compress tissue against said end effector
wherein said surgical apparatus is configured for the operative coupling to a source ultrasonic vibration such that said blade shaft transmits ultrasonic vibrations along its length to said end effector, and further wherein said clamp arm includes a force limiting element

2. The ultrasonic surgical apparatus of claim 1 wherein said force limiting element is a spring arranged to oppose excess force applied by distal movement of said outer tube.

3. The ultrasonic surgical apparatus of claim 1 wherein said force limiting element is a spring arranged to oppose excess force applied by distal movement of said inner member.

4. The ultrasonic surgical apparatus of claim 1 wherein said outer tube includes force limiting features

5. The ultrasonic surgical apparatus of claim 4 wherein a said force limiting features include notches which allow the outer tube to expand or contract when significant axial load is applied.

6. The ultrasonic surgical apparatus of claim 5 wherein said notches alternate.

7. The ultrasonic surgical apparatus of claim 6 wherein said notches form a spiral pattern.

8. An ultrasonic surgical apparatus, comprising;
a housing;
an outer tube carried by said housing, said outer tube having distal and proximal ends;
an inner member carried by said housing, said inner member having distal and proximal ends;
a blade member carried by said housing, said blade member having an elongate shaft and an end effector located at a distal end of the blade member, said blade shaft positioned at least partially within the interior of said outer tube;
a clamp arm connected to said outer tube adjacent the distal end of the blade member, said clamp arm configured for selective pivotal movement towards the end effector of said blade member such that, during use, pivotal movement of the clamp arm toward the end effector of the blade member may be used to compress tissue against said end effector;
a drive yoke operably connected to said clamp arm such that movement of said yoke results in pivotal movement of said clamp arm;
a force limiting element;
wherein said surgical apparatus is configured for the operative coupling to a source ultrasonic vibration such that said blade shaft transmits ultrasonic vibrations along its length to said end effector,
and further wherein said force limiting element is operably between said drive yolk and said housing.

9. The ultrasonic surgical apparatus of claim 8 wherein the force limiting is a compression spring.

10. An ultrasonic surgical apparatus, comprising;
a housing;
an outer tube carried by said housing, said outer tube having distal and proximal ends;
an inner member carried by said housing, said inner member having distal and proximal ends;
a blade member carried by said housing, said blade member having an elongate shaft and an end effector located at a distal end of the blade member, said blade shaft positioned at least partially within the interior of said outer tube;
a clamp arm connected to said outer tube adjacent the distal end of the blade member, said clamp arm configured for selective pivotal movement towards the end effector of said blade member such that, during use, pivotal movement of the clamp arm toward the end effector of the blade member may be used to compress tissue against said end effector;
a linkage operably connected to said clamp arm such that movement of said yoke results in pivotal movement of said clamp arm;
a force limiting element;
wherein said surgical apparatus is configured for the operative coupling to a source ultrasonic vibration such that said blade shaft transmits ultrasonic vibrations along its length to said end effector,
and further wherein said force limiting element is operably between said linkage and said housing.

11. The ultrasonic surgical apparatus of claim 10 wherein the force limiting element between the linkage and housing is a compression spring.

12. The ultrasonic surgical apparatus of claim 1 wherein the force limiting element is deformable.

13. The ultrasonic surgical apparatus of claim 10 wherein the force limiting element between the linkage and housing is a cantilever spring.

14. The ultrasonic surgical apparatus of claim 10 wherein the force limiting element between the linkage and housing is a gas spring.

15. The ultrasonic surgical apparatus of claim 10 wherein the force limiting element between the linkage and housing is a Belleville washer.

16. The ultrasonic surgical apparatus of claim 11 wherein the compression spring is a volute spring.

17. The ultrasonic surgical apparatus of claim 10 wherein the force limiting element is comprised of one or more of the following types of springs: coil, helical, conical, volute, leaf, V-spring, disc, constant-force, mainspring, elastomeric, washer, torsion, extension, or wave.
